(19) United States
(12) Patent Application Publication
(10) Pub. No.: US 2017/0246048 A1
(43) Pub. Date: Aug. 31, 2017

(54) Disposable Training Diaper with Wetting-Point Vent

(71) Applicants: Thadora Houmas, Langhorne, PA (US); Kimberly A. Vinch, Lawenceville, NJ (US)

(72) Inventors: Thadora Houmas, Langhorne, PA (US); Kimberly A. Vinch, Lawenceville, NJ (US)

(21) Appl. No.: 15/052,887

(22) Filed: Feb. 25, 2016

(51) Int. Cl. A61F 13/496; A61F 13/49

(52) U.S. Cl. CPC A61F 13/496; A61F 13/49058; A61F 2013/49098

(57) ABSTRACT

A disposable diaper especially constructed for the purpose of toilet training is disclosed. The diaper has conventional disposable diaper construction but also incorporates a wetting point vent that is an opening located so as to provide unimpeded egress of urine. This allow the diaper to be used as a step in a toilet training protocol in which the user, comfortable urinating in a diaper, is provided the comforting sensation of wearing a diaper while being acclimated to urinating in a toilet. Once acclimated to urinating in a toilet, the diaper is removed, and the user fully toilet trained. In a variant, the training diaper has a flap that covers the vent during normal use, but may be folded back reveal the vent during toilet training. In a further variant, there is a removable cover that conceals the vent during normal use but is removed for toilet training.

### BACKGROUND OF THE INVENTION

(1) Field of the Invention

The present invention is directed to the technical field of hygiene and more particularly to articles intended to be in contact with the human skin and absorb excretions from the natural genital and anal openings, and most particularly to disposable diapers especially constructed for the purpose of toilet training.

(2) Description of Related Art

The technical problem of toilet training is inherent in the technical fields of child-rearing and hygiene. Using a toilet is a learnt behavior that can usually be taught once a child is physically and emotionally ready, typically starting around the age of two. Physical readiness includes factors such as the ability to follow basic directions as well as the ability to sit on, and rise off, the toilet chair. Emotional factors may include an interest in toileting and discomfort, or dissatisfaction, with wearing diapers.

Toilet training is essentially a form of behavior modification that may be implemented in a number of steps.

A first step may, for instance, be to familiarize the child with the toilet, or potty-chair, and its purpose, either by having them observe other family members using it, or by demonstrations such as emptying dirty diapers into it.

A next step may be to seat the child on the toilet for a few minutes at a time, several times a day, particularly when they show signs of wanting to urinate or defecate. Rewarding success with positive reinforcement is advised.

The goal is for the child to learn correct toilet behavior by positively and correctly associating the toilet, the urges to urinate and defecate, and the physical actions of sitting on the toilet and voiding the bladder or bowel.

Effective daytime toilet training may be defined as a child having less than four wetting accidents per week. The vast majority, about 98%, of children in the United States achieve this level by their third birthday. There are, however, significant populations, including autistic and developmentally delayed children, for which toilet training may be a more significant developmental challenge. For these groups, a lack of toileting skills may impede their development by limiting their independence and diminishing their dignity in social domains. School aged children that are diagnosed as autistic, or are developmentally delayed, may, therefore, require toilet training within the school.

Behavioral analysts have developed toilet training protocols that may be implemented in school environments as described in detail in, for instance, “Toilet Training Children with Autism and Developmental Delays: An Effective Program for School Settings” by Michael A. Cocchiola et al, Behavior Analysis in Practice, 5(2): pp 60-64, 2012, the contents of which are hereby incorporated by reference in their entirety.

However, even such carefully thought-out procedures typically take about three-months of careful implementation to succeed.

The present invention addresses these problems by means of a novel disposable diaper that is described in greater detail below, and which may be use to facilitate more rapid toilet training.

The relevant prior art addressing the technical problem articulated above includes:

U.S. Pat. No. 9,186,279 issued to Sakaguchi et al. on Nov. 17, 2015 entitled “Disposable diaper” that describes a disposable diaper includes: a main body having a liquid-permeable top sheet, a liquid-impermeable outer sheet an absorber provided between the top sheet and the outer sheet, and a gather. The gather includes a plurality of elastic members arranged in a width direction outboard of the absorber. The elastic members are fixed to the gather in a state where the elastic members are stretched in a longitudinal direction. A side flap is located in one of end portions of the main body in the longitudinal direction and projects outwardly beyond at least one of end portions of the main body in the width direction. A joint portion joins the side flap and the main body. In a spread-out state of the disposable diaper, an outermost elastic member among the elastic members is arranged outboard of the joint portion in the width direction.

U.S. Pat. No. 8,992,495 issued to Howell on Mar. 31, 2015 entitled “Wrap-around canine utility harness with integral diaper” that describes a wrap-around canine utility harness with integral diaper comprises a body wrap utility harness apparatus with a detachable integral diaper. The harness apparatus may secure the integral diaper covering the anus and urethra while still maintaining utility and protection as a dog control harness. The harness apparatus is comprised of a fabric wrap in two perpendicularly connected sections. A longitudinal wrap forms a continuous cover from a withers area caudally along the dorsal, wrapping around the posterior of the dog, cranially over the ventral ending near the upper chest. A perpendicularly attached trunk wrap section completely encircles trunk of the dog overlapping and attaching at the dorsal. The integral diaper extends from above the tail encircling the base of the tail, around the rump to a caudal-cranial midpoint on the ventral able to contain excrement and protect anus and urethra area for both male and female dogs.

U.S. Pat. No. 5,779,690 issued to Gustafsson et al. on Jul. 14, 1998 entitled “Diaper with opening in top sheet” that describes a diaper having a front part (12), a back part (14) and an intermediate crotch part (13) which includes an absorbent body (1) joined to a fluid-impermeable bottom sheet (7) and a fluid-permeable top sheet (9) which lies proximal to the wearer's body in use and which includes an opening (15) situated in the back and crotch part of the diaper and elastic devices (20, 21) and which is unattached to the absorbent body at least within the region of the opening (15). Two elastic devices (20, 21) in the top sheet (9) extend divergently in relation to one another, from the front edge (16) of the opening (15) in the crotch part (13) towards the back part (14) and on respective sides of the opening in the top sheet (9).

Various other implementations are known in the art, but, as far as can be reasonably ascertained from currently and readily available documentation, they fail to adequately address all of the problems solved by the invention described herein.

### BRIEF SUMMARY OF THE INVENTION

An inventive disposable training diaper having a wetting-point vent is disclosed.

The disposable training diaper of the present invention may, for instance, be used in a toilet training protocol. The steps of such a toilet training protocol may include introducing the user to toilet facilities while wearing the diaper having a wetting point vent. A problem with toilet training is that a child used to urinating while wearing a diaper, may feel very uncomfortable when seated diaper-less on a toilet and consequently refuse to urinate. The disposable training diaper with a wetting-point vent allows the user to experience the reassuring comfort of wearing the diaper while seated on the toilet. The wearer of such a diaper may, therefore, urinate while seated on the toilet. These allows the user to be rewarded for correct behavior, and a positive association between sitting on the toilet and urinating to begin to be established. Once the association between sitting on the toilet and urinating has been firmly established, the training diaper may be dispensed with and the user may now transition to urinating while seated diaper-less on the toilet.

In a preferred embodiment, the disposable training diaper may be constructed to have a first outer diaper-layer made of a liquid-permeable material and a second outer diaper-layer made of a liquid-impermeable material with an intermediate diaper-layer of a water-absorbing material sandwiched between the two outer layers. The outer diaper-layer made from liquid permeable material is the layer that may abut the user's body when the diaper is worn. The disposable training diaper may also have a wetting-point vent that may be an opening located so as to provide unimpeded egress to urine discharged by the user when the training diaper is correctly positioned on the user.

Although it may seem counter-intuitive to provide a diaper that does not stop or capture urine, an aim of the invention is to provide the user with the comfortable feeling of wearing a diaper while being able to urinate into a toilet for the purpose of toilet training. That the training diaper need not be particularly water absorbing may allow the intermediate diaper-layer of a water-absorbing material to be made of less expensive material than the super absorbent polymer mixture typically used in diapers, so long as the feel, or sensation, of wearing the diaper remains essentially the same, or similar, to that of a diaper the user is used to wearing.

In a further preferred embodiment of the invention, the disposable training diaper may incorporate either a flap, or a removable cover, either of which, when in place, may allow the training diaper to function as a conventional diaper and absorb any egress of urine, but when removed, or folded out of the way, may allow unimpeded egress of urine and, therefore, be of use in the toilet training protocol outlined in brief above.

Therefore, the present invention succeeds in conferring the following, and others not mentioned, desirable and useful benefits and objectives.

It is an object of the present invention to provide a diaper that may be used for effective toilet training.

It is another object of the present invention to provide a diaper that may be used both as a toilet training device and function as a conventional diaper.

### DESCRIPTION OF THE SEVERAL VIEWS OF THE DRAWINGS

FIG. 1 shows a schematic isometric view of a disposable training diaper of the present invention with a wetting-point vent fitted on a user.

FIG. 2A shows a schematic plan view of a disposable training diaper of the present invention with a wetting-point vent as seen from the side abutting the user when in use.

FIG. 2B shows a schematic side view of a disposable training diaper of the present invention with a wetting-point vent.

FIG. 3A shows a schematic isometric view of a disposable training diaper with flap-covered wetting-point vent of the present invention fitted on a user, with the flap held closed.

FIG. 3B shows a schematic isometric view of a disposable training diaper with a flap-covered wetting-point vent of the present invention fitted on a user, with the flap held open.

FIG. 4A shows a schematic plan view of a disposable training diaper with a flap-covered wetting-point vent of the present invention, as seen from the user non-abutting side, with the flap held closed.

FIG. 4B shows a schematic side view of a disposable training diaper with a flap-covered wetting-point vent of the present invention with the flap held closed.

FIG. 4C shows a schematic plan view of a disposable training diaper with a flap-covered wetting-point vent of the present invention, as seen from the user non-abutting side, with the flap held open.

FIG. 4D shows a schematic side view of a disposable training diaper with a flap-covered wetting-point vent of the present invention, with the flap held open.

FIG. 5A shows a schematic isometric view of a disposable training diaper with a removable wetting-point cover of the present invention, with the removable wetting-point cover held in place.

FIG. 5B shows a schematic isometric view of a disposable training diaper with a removable wetting-point cover of the present invention, with the removable wetting-point cover removed.

FIG. 6A shows a schematic plan view of a disposable training diaper with a removable wetting-point cover of the present invention, as seen from the user non-abutting side, with the removable wetting-point cover in place.

FIG. 6B shows a schematic side view of a disposable training diaper with a removable wetting-point cover of the present invention, with the cover in place.

FIG. 6C shows a schematic plan view of a disposable training diaper with a removable wetting-point cover of the present invention, as seen from the user non-abutting side, with the removable wetting-point cover removed.

FIG. 6D shows a schematic side view of a disposable training diaper with a removable wetting-point cover of the present invention, with the removable wetting-point cover removed.

FIG. 7 shows a schematic flow diagram of a process of using a disposable training diaper with a wetting-point vent for the purposes of toilet training a user.

### DETAILED DESCRIPTION OF THE INVENTION

The preferred embodiments of the present invention will now be described in more detail with reference to the drawings in which identical elements in the various figures are, as far as possible, identified with the same reference numerals. These embodiments are provided by way of explanation of the present invention, which is not, however, intended to be limited thereto. Those of ordinary skill in the art may appreciate upon reading the present specification and viewing the present drawings that various modifications and variations may be made thereto.

The disposable training diaper having a wetting-point vent may be used in toilet training users. It is, therefore, useful to outline some of the technical problems involved in typical toilet training.

Toilet training may be thought of as a form of behavior modification and may, therefore, rely to a significant extent on the implementation of established psychological principles such as, but not limited to, the positive reinforcement of desired behaviors. Positive reinforcement, however, depends on waiting for, or somehow eliciting, the desired behavior so that it can then be reinforced.

In a very simple form, toilet training is placing a child on an appropriately sized toilet when they are inclined to urinate or defecate and then rewarding them when they do. After a number of rewarded successes, the child associates correct toilet behavior with reward and the correct toilet behavior becomes a learnt skill. Once the skill is sufficiently learnt, the actual reward may be gradually withdrawn as the behavior itself becomes sufficient reward in of itself.

However, as stated previously, a major problem is getting a correct behavior to occur so that a reward can be given to start encouraging that behavior.

Toilet training children reared using disposable diapers can be problematic because, since the incorporation of Super Absorbent Polymer (SAP) fillings into the diapers in the late 1970's, disposable diapers have become very comfortable to wear even after they are soiled. Children feel comfortable urinating in their SAP filled disposable diapers, and may, therefore, not feel inclined to urinate without them on, especially when placed sitting diaper-less on an unfamiliar toilet seat that may also feel uncomfortable to sit on with their naked buttocks. This aversion to urinating while uncomfortable in an unfamiliar situation may be particularly strong in children diagnosed with autism who tend to have very rigid behavior patterns and also tend to be averse to unusual sensations such as their naked buttocks touching a toilet seat.

One method of overcoming the dilemma of eliciting the initial correct behavior is to enlist the behavior modification concept of shaping. Shaping is the development of a new behavior by the reinforcement of successive approximations to the desired end behavior, and the extinguishing of preceding approximations.

In the context of toilet training with the training diapers having a wetting-point vent of the present invention the shaping may be accomplished in the following two steps.

At the start, the child is comfortable urinating while wearing a disposable diaper.

The first approximation to correct toileting behavior may be to have them urinate while wearing a training diaper having a wetting-point vent. This allows them to still have the reassuring sensation of wearing their familiar diaper, while acclimating to urinating while sitting on the toilet. This may be done by fitting the child with such a diaper and placing them on the toilet seat. After a few attempts, the child may become comfortable with sitting on the toilet, and be willing to urinate since they still feel the comfort of the familiar diaper around them. When they do urinate in the toilet through the vent, they may be rewarded with appropriate reinforcements such as, but not limited to, positive comments, clapping, and edible or drinkable rewards, or some combination thereof. After a sufficient number of reinforced successes, typically eight to twelve, the child forms a positive association with sitting on the toilet and urinating there. Once that association is sufficiently formed, the child may then be placed on the toilet seat without a diaper. This transition is now smaller as the child already has a positive association between sitting on the toilet and urinating and may, therefore, be far more likely to now urinate even without the comfort of the diaper. Once such behavior occurs, it may be rewarded, and after a sufficient number of rewarded successes, the required toiling behavior may now have been learnt, and the prior behavior of wearing a diaper on the toilet may be extinguished, or faded out.

Although the discussion of toilet training above has been confined primarily to urinating, one of ordinary skill in the art will appreciate that the concepts may apply equally well to defecating.

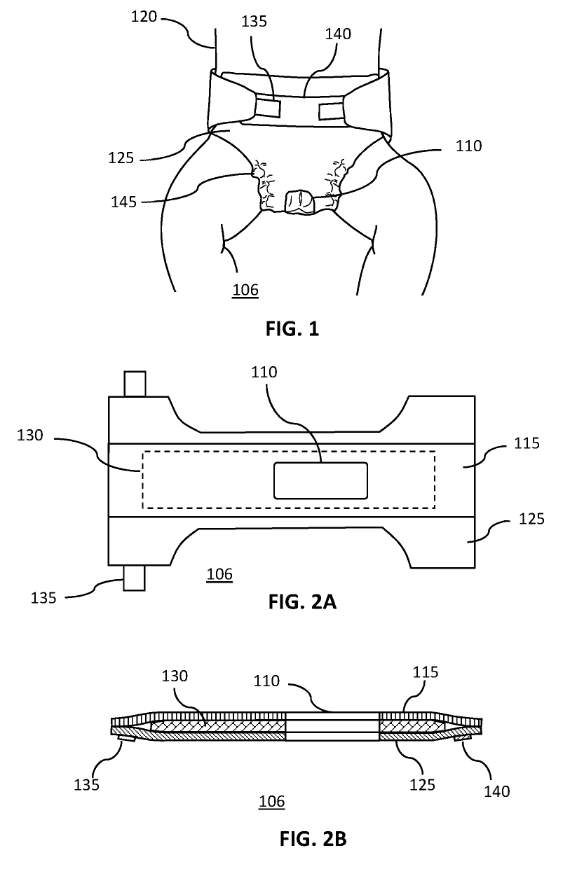

FIG. 1 shows a schematic isometric view of a disposable training diaper 106 of the present invention with a wetting-point vent 110 fitted on a user 120.

The disposable training diaper 106 with wetting-point vent may, for instance, be of conventional disposable diaper construction and may be held in place by one or more diaper-securing tabs 135 that may attach to a landing zone 140 for the diaper-fastening tab.

The diaper landing zone 140 for a diaper-fastening tab may be attached onto the outer diaper-layer 125. Disposable diapers typically also have elasticated leg cuffs 145 to better contain any liquid.

FIG. 2A shows a schematic plan view of a disposable training diaper of the present invention with a wetting-point vent as seen from the side abutting the user when in use.

The disposable training diaper 106 may, for instance, be constructed with a first outer diaper-layer 115 made of a liquid-permeable material. This may be the layer intended to abut the users body when the diaper is worn. There may then be a second outer diaper-layer 125 made of a liquid-impermeable material that may be intended to retain feces and urine. Between these two outer layers there may be an intermediate diaper-layer 130 of a water-absorbing material. In conventional disposable diapers this water-absorbing layer may be made of Super Absorbent Polymer (SAP) that may, for instance, be a mixture of fluff and sodium polyacrylate. Sodium polyacrylate has the chemical formula [—CH2—CH(CO2Na)-]n and is a material capable of absorbing 200 to 300 times it mass in water. Moreover, it retains the water as a gel, helping make even a soiled diaper feel comfortable. The fluff is typically made from wood pulp, though it may also or instead contain cotton or wheat/corn based materials. The role of the fluff may be to help distribute the fluid throughout the absorbing layer.

The disposable training diaper 106 of the present invention further includes a wetting-point vent 110. This wetting-point vent 110 may, for instance, be an opening located so as to provide unimpeded egress to urine discharged by the user when the training diaper is correctly position on the user. The wetting-point vent 110 may extend through all three of the diaper layers, i.e., through the second outer diaper-layer 125 made of a liquid-impermeable material, the intermediate diaper-layer 130 made of a water-absorbing material and the intermediate diaper-layer 130 made of a water-absorbing material.

FIG. 2B shows a schematic side view of a disposable training diaper 106 of the present invention with a wetting-point vent.

The disposable training diaper 106 with wetting-point vent 110 may be constructed in a similar manner to conventional disposable diapers having three layers, a first outer diaper-layer 115 of a liquid-permeable material, a second outer diaper-layer 125 of a liquid-impermeable material and an intermediate diaper-layer 130 of a water-absorbing material. There may also be one or more diaper-securing tab 135 and one or more landing zones 140 for a diaper-fastening tab.

The first outer diaper-layer 115 that abuts the users body, often termed the top sheet of a diaper, may be made of a liquid-permeable material. This is typically a nonwoven polypropylene fabric that may have additional surfactant treatments to improve its hydrophilic character.

The second outer diaper-layer 125, often termed the back layer, may be made of a liquid-impermeable material. This is typically a nonwoven polyethylene material that may have additional surfactant treatments to improve its hydrophobic character.

The intermediate diaper-layer 130 may be made of a water-absorbing material, typically a Super Absorbent Polymer (SAP) that may, for instance, be a mixture of wood chip fluff and sodium polyacrylate.

Because the disposable training diaper 106 with wetting-point vent 110 may not be used to absorb fluid, the intermediate diaper-layer 130 may be made of alternate materials that may not be as effective in absorbing fluid as SAP, but that may be cheaper or easier to manufacture or handle in the manufacturing process. Alternate materials may, for instance, include natural substances such as, but not limited to, wheat starch. The training diapers of the present invention may also use less SAP, or even no SAP, as the absorbing function of the diaper may be secondary to how they feel to the user wearing them. As long as the disposable training diaper 106 with wetting-point vent 110 provide a user with a similar, or identical, feel, or sensation of comfort, as that of a type of disposable diaper the user is accustomed to wearing, they may be adequate for their role in toilet training.

The three layers of the diaper may be joined by techniques such as, but not limited to, gluing, heating or ultrasonic welding or some combination thereof. The joining may, for instance, be between the first and second outer layers and may be around their periphery.

In a preferred embodiment, the first and second outer layers may also be joined around the periphery of the wetting-point vent 110.

The wetting-point vent 110 may be sized and shaped to so as to provide unimpeded egress to urine discharged by the user when said training diaper is correctly position on the user. This size and shape may, therefore, be adjusted to suit the physiology of the user. The size and shape of the wetting-point vent 110 may therefore vary depending on the gender or age, or both, of the intended user.

In a further preferred embodiment of the invention, the disposable training diaper 106 may have the vent extended so as to also allow unimpeded egress of feces. Training diapers may, for instance, be adjusted to allow unimpeded egress of urine alone, feces alone or a combination of urine and feces.

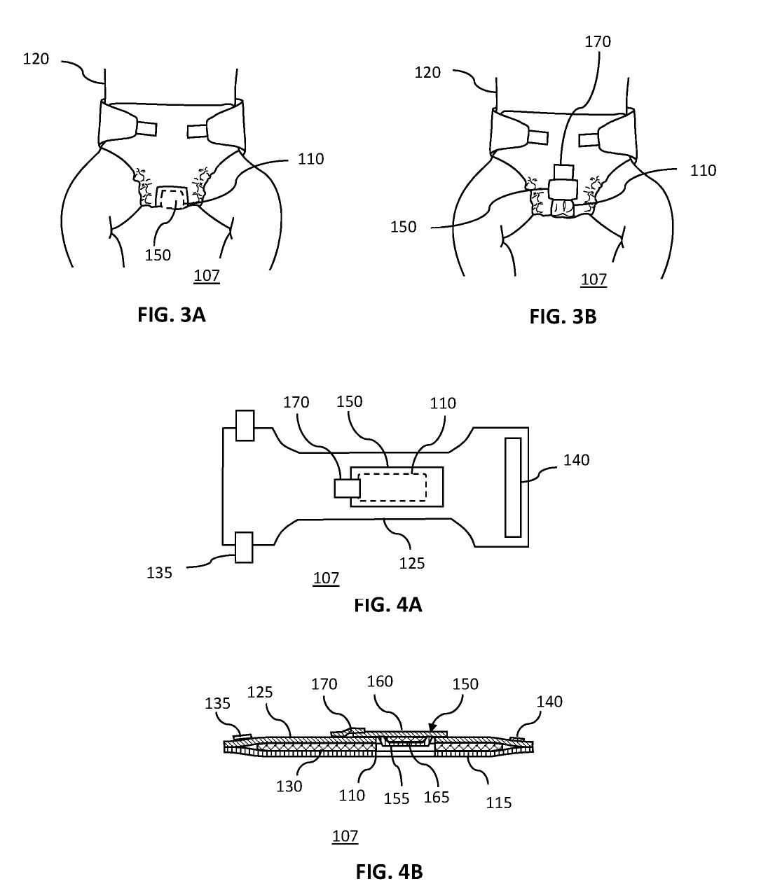

FIG. 3A shows a schematic isometric view of a disposable training diaper 107 with flap-covered wetting-point vent 110 of the present invention fitted on a user, with the flap 150 held closed. With the wetting-point flap 150 held in the closed position the disposable training diaper 107 with flap-covered wetting-point vent may now function as a conventional diaper.

FIG. 3B shows a schematic isometric view of a disposable training diaper 107 with a flap-covered wetting-point vent of the present invention fitted on a user, with the flap 150 held open. The wetting-point flap 150 may be held open by a flap-securing tab 170 that may function in a manner similar to the tabs used to hold the diaper to the user's body 120.

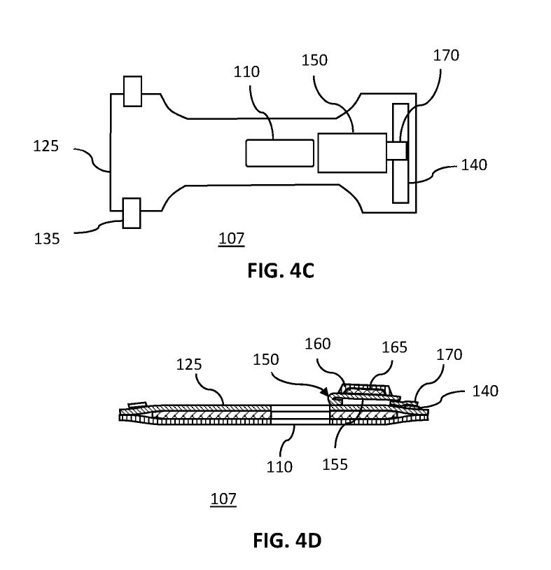

FIG. 4A shows a schematic plan view of a disposable training diaper 107 with a flap-covered wetting-point vent 110 of the present invention, with the flap 150 held closed, as viewed from the non-user-abutting side, i.e., the side not abutting the user when the diaper is being worn correctly.

The wetting-point flap 150 may be fixed at one end to the second outer diaper-layer 125 made of a liquid-impermeable material, and may be held in place at its other end by a flap-securing tab 170.

The flap-securing tab 170 may be of the same design as a typical diaper-securing tab 135. They may for instance have a hook-like surface structure that allows them to removably adhere to the second outer diaper-layer 125. They may also, or instead, be made of a material that is complementary in structure to the material of the diaper-fasting tab landing zone 140 thereby enabling a hook-and-loop type fasting arrangement.

FIG. 4B shows a schematic side view of a disposable training diaper 107 with a flap-covered wetting-point vent 110 of the present invention with the flap held closed.

The wetting-point flap 150 may have a construction similar to that of a conventional disposable diaper in that it includes a first outer flap-layer 155 of a liquid-permeable material, a second outer flap-layer 160 of a liquid-impermeable material and an intermediate flap-layer 165 of a water-absorbing material.

The first outer flap-layer 155 may, for instance, be made of the same liquid-permeable material as the first outer diaper-layer 115. This is typically a nonwoven polypropylene fabric that may have additional surfactant treatments to improve its hydrophilic character.

The second outer flap-layer 160, may, for instance, be made the same liquid-impermeable material as the second outer diaper-layer 125. This is typically a nonwoven polyethylene material that may have additional surfactant treatments to improve its hydrophobic character.

The intermediate flap-layer 165 may, for instance be made of the same water-absorbing material as the intermediate diaper-layer 130. This is typically a Super Absorbent Polymer (SAP) that may, for instance, be a mixture of wood chip fluff and sodium polyacrylate.

The first outer flap-layer 155 and the intermediate flap-layer 165 are preferably shaped and sized to be a close fit, or a push fit, to within the wetting-point vent 110.

The second outer flap-layer 160 is preferably shaped and sized to extend beyond the periphery of the wetting-point vent 110 on all edges.

In a preferred embodiment, the first and second outer flap-layers may be joined around the periphery of the first outer flap-layer 155. This joining may, for instance, be by a technique such as, but not limited to, gluing, heating or ultrasonic welding or some combination thereof.

In a first alignment, shown in FIG. 4B, the wetting-point flap 150 may be held in place by a flap-securing tab 170. The flap-securing tab 170 may function in a manner similar to manner in which the diaper-securing tab 135 and the diaper-fastening tab landing zone 140 function, i.e., as complementary components in a hook-and-loop fastening arrangement.

The flap-securing tab 170 may have attachment structures on both surfaces so as to attach both when closed and when open. The surface attachment structure of the flap-securing tab 170 may also, or instead, allow it to removably attach directly to the surface of the second outer diaper-layer 125.

In a further preferred embodiment of the invention, the flap-securing tab 170 may extend around the periphery of the second outer flap-layer 160. Such an extended, peripheral securing tab may provide a more secure attachment of the flap to the second outer diaper-layer 125 during use as a conventional diaper.

FIG. 4C shows a schematic plan view of a disposable training diaper 107 with a flap-covered wetting-point vent 110 of the present invention, as seen from the user non-abutting side, with the flap 150 held open.

The flap is shown being held open by the flap-securing tab 170 being attached to the landing zone 140 that may also be intended to hold a diaper-fastening tab. This may be achieved by the flap-securing tab 170 having the same surface structure as the diaper-securing tab 135. This surface structure may be such that the flap-securing tab 170 and the landing zone 140 act as complementary halves of a hook-and-loop fastener. The surface structure of the flap-securing tab 170 may also, or instead, be such that it may removably attach directly to the surface of the second outer diaper-layer 125.

FIG. 4D shows a schematic side view of a disposable training diaper 107 with a flap-covered wetting-point vent 110 of the present invention, with the flap 150 held open.

In the second alignment, shown in FIG. 4D, the wetting-point flap 150 has been folded back about an upper-edge by which it is fixedly attached to, or is a part of, the second outer diaper-layer 125. As shown in FIG. 4D, it may be held in place by the flap-securing tab 170 being attached to the landing zone 140 for a diaper-fastening tab.

In this second alignment, the wetting-point vent 110 is clear, and egress of urine is unimpeded, thereby facilitating the device's use as a training diaper.

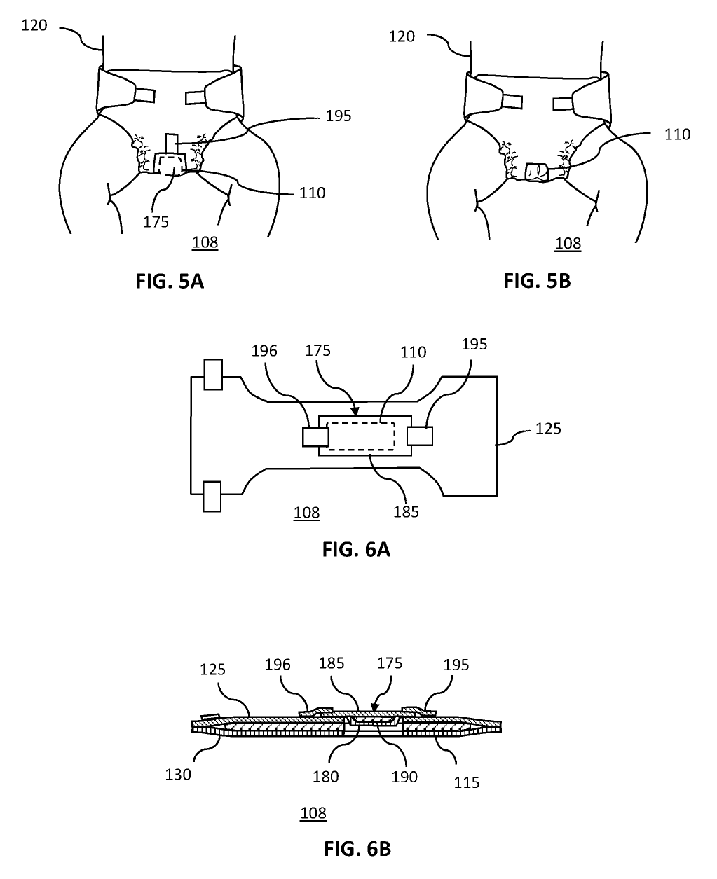

FIG. 5A shows a schematic isometric view of a disposable training diaper 108 with a removable wetting-point cover 175 of the present invention, fitted to a user's body 120, with the removable wetting-point cover 175 held in place.

As shown in FIG. 5A, the removable wetting-point cover 175 may be held in place over the wetting-point vent 110 by means of a upper cover attachment-tab 195. The upper cover attachment-tab 195 may have surface structure enabling it to adhere to the outer surface of the diaper. This ability to adhere may, for instance, be that of a hook-and-loop fastener.

With the removable wetting-point cover 175 held in place over the wetting-point vent 110 the disposable training diaper 108 may function as a conventional disposable diaper.

FIG. 5B shows a schematic isometric view of a disposable training diaper 108 with a removable wetting-point cover of the present invention, fitted to a user's body 120, with the removable wetting-point cover removed. The wetting-point vent 110 may be sized and shaped so that without the cover, egress of urine is unimpeded and the disposable training diaper 108 may be used for toilet training using, for instance, the toilet training protocol described above.

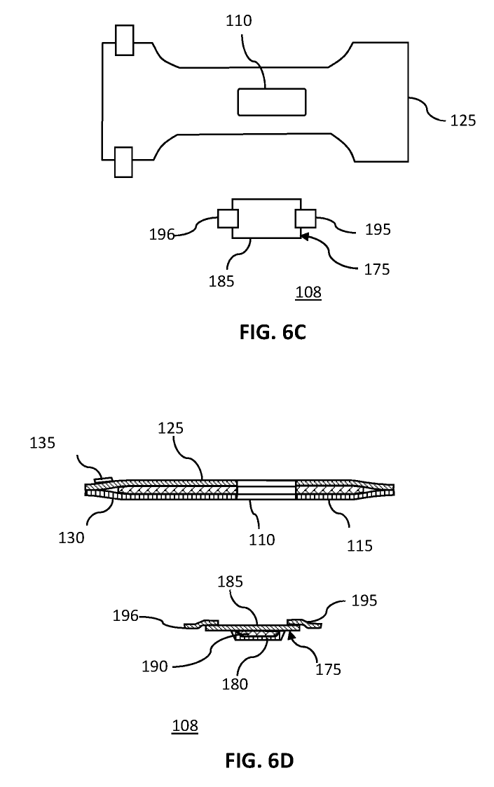

FIG. 6A shows a schematic plan view of a disposable training diaper 108 with a removable wetting-point cover of the present invention, as seen from the user non-abutting side, with the removable wetting-point cover 175 in place.

The removable wetting-point cover 175 may be held in place covering the wetting-point vent 110 by one or more tabs, such as the upper cover attachment-tab 195 and the lower cover attachment-tab 196. The cover attachment tabs may, for instance, function as hook-and-loop fasters by, for instance, having a surface structure that is complementary to a surface structure on at least a portion of the second outer diaper-layer 125.

The upper cover attachment-tab 195 and the lower cover attachment-tab 196 may both be rigidly attached to, or a part of, the second outer cover-layer 185.

In a further preferred embodiment of the invention, the cover attachment-tabs may also extend around and out from the periphery of the second outer cover-layer 185. Such an arrangement may, for instance, ensure that the removable wetting-point cover 175 is held more securely so the disposable training diaper 108 may fulfill its fluid absorbing functions better.

FIG. 6B shows a schematic side view of a disposable training diaper 108 with a removable wetting-point cover 175 of the present invention, with the cover in place.

The removable wetting-point cover 175 may have a construction similar to that of a conventional disposable diaper in that it includes a first outer cover-layer 180 of a liquid-permeable material, a second outer cover-layer 185 of a liquid-impermeable material and an intermediate layer 190 of a water-absorbing material of a water-absorbing material.

The first outer cover-layer 180 may, for instance, be made of the same liquid-permeable material as the first outer diaper-layer 115. This is typically a nonwoven polypropylene fabric that may have additional surfactant treatments to improve its hydrophilic character.

The second outer cover-layer 185 may, for instance, be made the same liquid-impermeable material as the second outer diaper-layer 125. This is typically a nonwoven polyethylene material that may have additional surfactant treatments to improve its hydrophobic character.

The intermediate layer 190 may, for instance be made of the same water-absorbing material as the intermediate diaper-layer 130. This is typically a Super Absorbent Polymer (SAP) that may, for instance, be a mixture of wood chip fluff and sodium polyacrylate.

The first outer cover-layer 180 and the intermediate layer 190 are preferably shaped and sized to be a close, or push fit, to within the wetting-point vent 110.

The second outer cover-layer 185 is preferably shaped and sized to extend beyond the periphery of the wetting-point vent 110 on all edges.

In a preferred embodiment, the first and second outer cover-layers may be joined around the periphery of the first outer cover-layer 180. This joining may, for instance, be by a technique such as, but not limited to, gluing, heating or ultrasonic welding or some combination thereof.

As shown in FIG. 6B, the removable wetting-point cover 175 may be held in place by an upper cover attachment-tab 195 and a lower cover attachment-tab 196. The cover attachment tabs may function in a manner similar to manner in which the diaper-securing tab and the diaper-fastening tab landing zone function, i.e., as complementary components in a hook-and-loop fastening arrangement. The surface attachment structure of the cover attachment tabs 195, 196 may also, or instead, allow it to removably attach directly to the surface of the second outer diaper-layer 125.

In a further preferred embodiment of the invention, the cover attachment tabs may extend around the periphery of the second outer cover-layer 185. Such an extended, peripheral cover attachment tab may provide a more secure attachment of the cover to the second outer diaper-layer 125 during use as a conventional diaper.

FIG. 6C shows a schematic plan view of a disposable training diaper 108 with a removable wetting-point cover 175 of the present invention, as seen from the user non-abutting side, with the removable wetting-point cover 175 removed.

As shown in FIG. 6C, there is the second outer diaper-layer 125 that may be made of a liquid-impermeable material and a wetting-point vent 110. The removable wetting-point cover 175 having a second outer cover-layer 185 of a liquid-impermeable material and a lower cover attachment-tab 196 and a upper cover attachment-tab 195 is shown moved away from the part of the diaper that may be fitted directly to a user's body.

FIG. 6D shows a schematic side view of a disposable training diaper 108 with a removable wetting-point cover 175 of the present invention, with the removable wetting-point cover 175 removed.

The portion of the diaper that may be fitted directly to a user's body is shown having a first outer diaper-layer 115 of a liquid-permeable material, a second outer diaper-layer 125 of a liquid-impermeable material and an intermediate diaper-layer 130 of a water-absorbing material sandwiched between them. The wetting-point vent 110 may extend through all three layers, thereby allowing unimpeded egress of urine while being worn without the removable wetting-point cover 175 being attached, thereby enabling its use for toilet training in, for instance, the toilet training protocols described above.

The removable wetting-point cover 175 is shown in FIG. 6D removed from the portion of the diaper that may be attached directly to a user's body.

The removable wetting-point cover 175 may be constructed of a first outer cover-layer 180 made of a liquid-permeable material, a second outer cover-layer 185 made of a liquid-impermeable material, and an intermediate layer 190 made of a water-absorbing material, and sandwiched between the two outer layers.

The removable wetting-point cover 175 may also have an upper cover attachment-tab 195 and a lower cover attachment-tab 196 that may, for instance, function as a part of a hook-and-loop faster with the outer surface of the second outer diaper-layer 125, or a portion thereof, acting as the complementary part of the fastener.

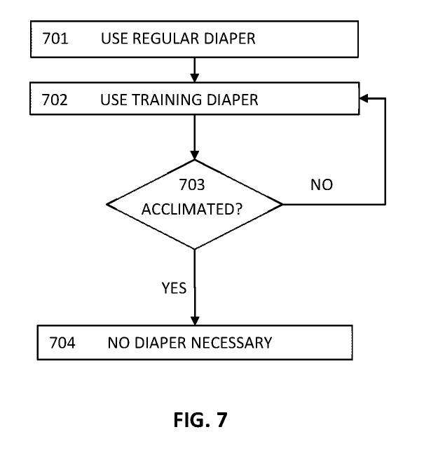

FIG. 7 shows a schematic flow diagram of a process of using a disposable training diaper with a wetting-point vent for the purposes of toilet training a user.

In Step 701, a user may have become accustomed to wearing a disposable diaper and to urinating in the disposable diaper while wearing it.

In Step 702, the user may now be fitted with a disposable training diaper of the present invention, having a wetting-point vent. Training may now take the form of placing the user equipped with the training diaper on a toilet seat either at regular, predetermined intervals, or when the user appears about to urinate. When the user urinates while on the toilet, they may be rewarded so as to reinforce that behavior.

In Step 703, a determination is made as to whether the user is acclimated, i.e., as to whether the user has learnt sufficient of a positive association between seated on the toilet and the act of urinating. This may, for instance, take the form of having recorded a sufficient number of rewarded instances of the desired toileting behavior, that may, for instance, be in a range of eight to twelve instances.

If the user is not deemed sufficiently acclimated, the training may return back to Step 702 and continue the use of the training diaper.

If the user is deemed sufficiently acclimated, the training may then proceed to Step 704 in which no diaper is worn when on the toilet. When the user urinates on the toilet with no diaper, they may be rewarded. After a sufficient number, typically in a range of eight to twelve, of rewarded successes of urinating without a diaper, the user may be deemed toilet trained. If problems urinating without a diaper persist, or return, the user may need to return to Step 702.

Although this invention has been described with a certain degree of particularity, it is to be understood that the present disclosure has been made only by way of illustration and that numerous changes in the details of construction and arrangement of parts may be resorted to without departing from the spirit and the scope of the invention.

1. A disposable training diaper having a rectangular wetting-point vent, comprising:
a first outer diaper-layer, comprised of a liquid-permeable material intended to abut the user's body;
a second outer diaper-layer, comprised of a liquid-impermeable material;
an intermediate diaper-layer, comprised of a water-absorbing material situated between said first and second layers, and having contiguous surface-contact with both said first and second outer diaper-layers;
a wetting-point flap, comprising:
a first outer flap-layer, comprised of a liquid-permeable material, and sized and shaped to fit within said rectangular wetting point vent;
a second outer flap-layer, comprised of a liquid-impermeable material, fixed at an upper-end of said second outer flap-layer to said second outer diaper-layer, and sized and shaped to extend beyond a periphery of said rectangular wetting-point vent;
an intermediate flap-layer, comprised of a water-absorbing material, situated between said first and second outer flap-layers, having contiguous surface-contact with both said first and second outer flap-layers, and sized and shaped to be a push-fit within said rectangular wetting-point vent; and
a flap-securing tab located at a lower-end of said second outer flap-layer, said flap-securing tab having a first attachment structures on a lower surface and a second attachment structure on an upper surface;
a landing zone located at an upper external portion of said second outer diaper-layer, having a complementary hook and loop structure to both said second attachment structure on said upper surface of said flap-securing tab and to an attachment structure on a diaper securing tab, and wherein said landing zone is situated such that it serves to secure both one or more diaper-securing tabs that hold said diaper in place on said user, and to secure said wetting point flap when said user requires unimpeded egress of urine; and
wherein, said wetting-point vent comprises a rectangular opening with a length greater than twice the width, located so as to provide unimpeded egress to urine discharged by the user when said training diaper is correctly positioned on the user and uncovered, said wetting-point vent extending through said first and second outer diaper-layers and through said intermediate diaper-layer; and,
wherein, in a first alignment, said wetting-point flap covers said rectangular wetting-point vent and is attached by an attachment structure on a first side of said flap-securing tab to said second outer diaper-layer, thereby preventing egress of urine discharged by the user; and
in a second alignment, said wetting-point flap is folded-back and attached by a second, opposite side of said flap-securing tab to a said landing zone in a secure manner such that said rectangular wetting-point vent is uncovered, so as to provide unimpeded egress to urine discharged by the user when said training diaper is correctly positioned on the user.

2-3. (canceled)

4. The disposable training diaper of claim 1, wherein said first and second outer diaper-layers are sealed together at a periphery of said wetting-point vent.

5. The disposable training diaper of claim 1 wherein said first outer flap-layer of a liquid-permeable material and said second outer flap-layer are sealed together at a periphery of first outer flap-layer.

6. The disposable training diaper of claim 5, wherein aid flap-securing tab extends outward from, and around a periphery of said second outer flap-layer.

7. The disposable training diaper of claim 5, wherein said flap-securing tab is comprised of one of a hook or a loop material and said second outer diaper-layer comprises a region of complementary material thereby enabling a hook-and-loop fastening of said flap-securing tab to said second outer diaper-layer.

8-10. (canceled)
